(19) United States
(12) Patent Application Publication
(10) Pub. No.: US 2016/0313321 A1
(43) Pub. Date: Oct. 27, 2016

(54) DIAGNOSTIC TEST STRIPS WITH FLASH MEMORY DEVICES AND METHODS OF USE THEREFORE

(71) Applicants: Ted Titmus, Mission Viejo, CA (US); William Pat Price, Henderson, NV (US)

(72) Inventors: Ted Titmus, Mission Viejo, CA (US); William Pat Price, Henderson, NV (US)

(21) Appl. No.: 14/694,916

(22) Filed: Apr. 23, 2015

(51) Int. Cl. G01N 33/543; G06F 13/42

(52) U.S. Cl. CPC G01N 33/54386; G06F 13/4282

(57) ABSTRACT

Some embodiments provide for a diagnostic test strip having a carrier strip that contains a region with one or more test pads in contact with the carrier strip and another region that has one or more memory devices in contact with the carrier strip. The one or more memory devices have connecter pins in contact with the carrier strip and they are optionally in electrical contact with each other when more than one memory device is utilized. Other embodiments provide for a method of detecting one or more analytes in a sample, the method involving contacting an embodiment of a diagnostic test strip with a sample such that the sample contacts the one or more test pads of the diagnostic test strip, and reading the results from the test strip.

### BACKGROUND OF THE INVENTION

1. Field of the Invention

The invention generally relates to diagnostic assay materials. More specifically, the invention relates to diagnostic test strips that produce and store information related to analyte presence, absence, and/or concentration and methods for the use of said diagnostic test strips.

2. Description of the Related Art

Many types of assays have been used to detect the presence of various substances, generally referred to as analytes, in physiological fluids such as urine. These assays often involve antigen-antibody reactions; synthetic conjugates comprising radioactive, enzymatic, fluorescent, or visually observable metal sol tags; and specially designed reactor chambers. In all these assays, there is a receptor; e.g., an antibody or chemical, which is specific for the selected analyte; and a means for detecting the presence, and often the amount, of the analyte. While some tests are designed to make a quantitative determination, in many circumstances all that is required is a qualitative positive/negative indication. However, in some circumstances the analyte of interest is present in the test sample in very small concentrations. Such circumstances require an assay to be very sensitive in order to detect the presence, absence, and/or concentration of the desired analyte. False positives and false negatives for qualitative assays can also be problematic.

Rapid, accurate, and reproducible analyte detection is important in many technological fields and applications. This is especially true for law enforcement officials, physicians, aid workers, employers, parents, and test subjects because safety and livelihood could depend upon the presence, absence, and/or concentration of certain analytes. In some circumstances, untrained individuals may need to test for the presence of an analyte to determine personal safety and health. In other circumstances, analyte detection is important for maintaining accurate records that are admissible in court. What is needed is a simple, accurate assay that provides trustworthy recordation of the presence, absence, and/or concentration of one or more analytes. These and other objects and features of the invention will be apparent from the following description, drawings, and claims.

### SUMMARY OF THE INVENTION

Provided herein are diagnostic assay materials. More specifically, diagnostic test strips are disclosed which produce and store information related to analyte presence, absence, and/or concentration and methods for the use of said diagnostic test strips. From this description, in conjunction with other items, the advantages of the invention will become clear and apparent more so based upon the hereinafter descriptions and claims, which are supported by drawings with numbers relating to parts, wherein are described in the following sections containing the relating numbers.

In one aspect of the invention, a diagnostic test strip is provided. The test strip may include a carrier strip. The carrier strip may further include a first region of the test strip with one or more test pads in contact with the carrier strip and a second region containing one or more flash memory devices in contact with the carrier strip. The test strip may further include connecter pins in contact with the carrier strip and they may be in electrical contact with each other when more than one memory device is utilized. Advantageously, the first and second region are not in fluid communication with each other.

In another aspect of the invention, the flash memory devices may be read-only devices. Alternatively, the flash memory devices may be rewritable devices. Optionally, the test strip further includes two or more flash memory devices, wherein at least one device is a read-only device and the other is a rewritable device.

In yet another aspect of the invention, the diagnostic test strip may further include a network controller device in contact with the carrier strip and in electrical contact with the one or more flash memory devices and the connector pins. Optionally, the connector pins are bus connector pins, USB data bus, serial data bus, or a 1394 data bus. The connector pins may be adapted for an Ethernet connection.

The one or more write-once memory device may contain information about the manufacture date, manufacturer, and the identifying characteristics of any test reagents contained on the one or more test pads. Advantageously, the test pads include a test reagent. The diagnostic test strip may include at least two or more test pads each with a different test reagent and each reagent is capable of testing for a different marker on the same analyte. In some embodiments, the at least one test pad further includes a signaling reagent.

In another aspect of the invention, the diagnostic test strip comprises at least one test pad containing a reagent that tests for a serum-borne analyte. The at least one test pad may include a reagent that tests for a plasma-borne analyte, a blood borne analyte, a urine-borne analyte, a semen-borne analyte, ascites-borne analyte, or a cerebral spinal fluid-borne analyte.

Other embodiments provide for a method of detecting one or more analytes in a sample, the method involving contacting a diagnostic test strip with a sample such that the sample contacts the one or more test pads of the diagnostic test strip, and reading the results from the test strip. The method may further include contacting the test strip with one or more signaling reagents so that the one or more reagents contact the one or more test pads.

Optionally, the method may include recording the results from the test strip and the information read from the memory in a database. The memory may be an RF device, and reading the information is performed with an RF device. The information read from the memory may include information identifying the diagnostic test strip. Recording the results can be accomplished by taking a picture of the test pads with a camera, and saving the picture in the database. Advantageously, the method may also include storing additional information in the database, the additional information being related to one or more of a time of the contacting the diagnostic test strip with the sample, a date of the contacting the diagnostic test strip with the sample, a location where contacting the diagnostic test strip with the sample, an identification of the source of the sample, and identification of an individual administering the detecting method, demographic data of the source of the sample, and physiological data of the source of the sample. Results may be electronically recorded from the test strip and stored in the memory. Electronically recording the results from the test strip may include taking a picture of the test pads with a camera.

In another aspect of the invention, the method includes storing additional information in the memory, the additional information being related to one or more of a time of the contacting the diagnostic test strip with the sample, a date of the contacting the diagnostic test strip with the sample, a location where contacting the diagnostic test strip with the sample, an identification of the source of the sample, and identification of an individual administering the detecting method, demographic data of the source of the sample, and physiological data of the source of the sample.

The patient sample may be serum, semen, or urine. In the case of urine, the test strip may be directly contacted with the patient's urine stream. The patient sample may be saliva, in which case, the test strip may be contacted with the patient's tongue. Alternatively, the patient sample may be blood and the test strip may be contacted directly with the source of the blood. Optionally, the patient sample may be ascites, sputum, or cerebral spinal fluid.

### BRIEF DESCRIPTION OF THE DRAWINGS

FIG. 1A is a top view of an embodiment of a diagnostic test strip having two test pads and a single memory device.

FIG. 1B is a perspective view of an embodiment of a diagnostic test strip having two test pads and a single memory device.

FIG. 2A is a top view of an embodiment of a diagnostic test strip having two test pads and two memory devices.

FIG. 2B is a perspective view of an embodiment of a diagnostic test strip having two test pads and two memory devices.

FIG. 3A is a top view of an embodiment of a diagnostic test strip having two test pads and three memory devices.

FIG. 3B is a perspective view of an embodiment of a diagnostic test strip having two test pads and three memory devices.

FIG. 3C is an expanded view of an embodiment of a circular test pad having two test pad layers.

FIG. 3D is a perspective view of the test pads stacked to produce a “plus” sign.

FIG. 4A is a top view of an embodiment of a diagnostic test strip.

FIG. 4B is a perspective view of diagnostic test strip.

FIG. 4C is an exploded view of a test pad.

FIG. 4D is a perspective view of the test pad of FIG. 4C.

### DETAILED DESCRIPTION

The present application relates to U.S. patent application Ser. No. ______, filed ______ entitled “DIAGNOSTIC TEST STRIPS WITH MULTIPLE LAMINATED LAYERS CONTAINING ONE OR MORE REAGENT-CARRYING PADS IN ONE OR MORE LAYERS”, Attorney Docket Number TTUSA.005A2, U.S. patent application Ser. No. ______, filed ______ entitled “MECHANICAL ATTACHMENT OF TEST PADS TO A DIAGNOSTIC TEST STRIP”, Attorney Docket Number TTUSA.006A2, U.S. patent application Ser. No. ______, filed ______ entitled “MECHANICAL ATTACHMENT OF TEST PADS TO A DIAGNOSTIC TEST DEVICE”, Attorney Docket Number TTUSA.007A2, U.S. patent application Ser. No. ______, filed ______ entitled “DIAGNOSTIC TEST STRIP WITH SELF-ATTACHING TEST PADS AND METHODS OF USE THEREFORE”, Attorney Docket Number TTUSA.008A2, U.S. patent application Ser. No. ______, filed ______ entitled “DIAGNOSTIC TEST STRIP FOR ORAL SAMPLES AND METHOD OF USE THEREFORE”, Attorney Docket Number TTUSA.010A2, U.S. patent application Ser. No. ______, filed ______ entitled “DIAGNOSTIC TEST STRIPS HAVING ONE OR MORE TEST PAD LAYERS AND METHOD OF USE THEREFORE, Attorney Docket Number TTUSA.011A2, U.S. patent application Ser. No. ______, filed ______ entitled “SINGLE USE MEDICAL TEST PACKAGING”, Attorney Docket Number TTUSA.012A2, U.S. patent application Ser. No. ______, filed ______ entitled “DIAGNOSTIC TEST STRIPS FOR DETECTION OF PAST OR PRESENT INFECTION OF VARIOUS STRAINS OF HEPATITIS” Attorney Docket Number TTUSA.013A2, and U.S. patent application Ser. No. ______ filed ______ entitled “DIAGNOSTIC TEST STRIPS FOR DETECTION OF PRE-SPECIFIED BLOOD ALCOHOL LEVEL” Attorney Docket Number TTUSA.014A2, all of whom have the inventors Ted Titmus and William Pat Price, all of which are filed herewith this even date, all of the disclosures of which are hereby expressly incorporated by reference in their entirety and are hereby expressly made a portion of this application.

Features of the present disclosure will become more fully apparent from the following description and appended claims, taken in conjunction with the accompanying drawings. It will be understood these drawings depict only certain embodiments in accordance with the disclosure and, therefore, are not to be considered limiting of its scope; the disclosure will be described with additional specificity and detail through use of the accompanying drawings. Descriptions of unnecessary parts or elements may be omitted for clarity and conciseness, and like reference numerals refer to like elements throughout. In the drawings, the size and thickness of layers and regions may be exaggerated for clarity and convenience. An apparatus, system or method according to some of the described embodiments can have several aspects, no single one of which necessarily is solely responsible for the desirable attributes of the apparatus, system or method. After considering this discussion, and particularly after reading the section entitled “Detailed Description” one will understand how illustrated features serve to explain certain principles of the present disclosure.

Some embodiments of the invention provide for a diagnostic test strip having a carrier strip that contains a region with one or more test pads. The one or more test pads optionally contain test reagents and/or signaling reagents. The one or more test pads are in contact with the carrier strip and another region that has one or more memory devices also in contact with the carrier strip. The one or more memory devices have connecter pins in contact with the carrier strip and are optionally in electrical contact with each other. Additionally, the one or more memory devices are in electrical contact, either directly or indirectly, with the region containing the one or more test pads. However, the region containing the one or more memory devices is not in fluid communication with the region containing the one or more test pads. In some embodiments, the memory one or more devices include an RF memory.

As disclosed below, various features of the embodiments and methods of using the embodiments enable both trained and untrained personnel to reliably detect the presence, absence, and/or concentration of one or more analytes in a sample. Indeed, features of the embodiments and methods for their use allow for the detection of extremely small quantities of one or more particular analytes while avoiding false positives and false negatives. Furthermore, features of the embodiments and methods for their use allow for accurate and trustworthy storage of information related to the tested sample. Optionally, embodiments may both produce a signal that communicates information to the user and store information related to the test sample in an embodiment's one or more memory devices. Consequently, the invention is ideal for use in both prescription and over-the-counter assay test kits which will enable a consumer to self diagnose themselves and others, or test food and/or water prior to consumption.

It is further envisioned that in certain circumstances a user may benefit from not seeing or knowing the results of a certain sample's test. Examples of such circumstances include, but are not limited to, police investigations, detecting stigma-associated diseases, performing tests in situations where the sample provider may respond violently, or even medical and academic studies where information is blinded from sample providers and/or study coordinators. In these and other circumstances, information related to the test is stored in the one or more flash memory devices for access at a later time. Furthermore, the one or more memory devices may be encrypted to limit the access of certain individuals to the test data stored within. With such circumstances in mind, embodiments are envisioned that may not produce a signal such that a user can acquire information concerning the tested sample at or near the time of testing.

Embodiments of the invention can be used to detect any analyte which has heretofore been assayed using known immunoassay procedures, or known to be detectable by such procedures. Furthermore, it is envisioned that known methods can be modified as needed to afford suitable test reagents and/or signaling reagents that will detect analytes that are similar to analytes that have been previously detected using known procedures.

Some embodiments of the invention provide for a method of detecting one or more analytes in a sample, the method involving contacting an embodiment of a diagnostic test strip with a sample such that the sample contacts the one or more test pads of the diagnostic test strip, and reading the results from the test strip. Any method may involve detecting analytes from any of the following samples: physiological fluids such as mucus, blood, serum, blood plasma, lymph, puss, urine, feces, cerebral spinal fluid, ocular lens liquid, ascites, semen, sputum, and saliva; synthetic chemicals; water; air; food; and soil. In some circumstances, it may be advantageous to contact the diagnostic test strip with one or more signaling reagents such that the one or more signaling reagents contact the one or more test pads. For example, the analyte of interest and/or sample analyzed may require the contact of the one or more signaling reagents with the one or more test pads such that signal generation is facilitated and/or enhanced.

Any method's results may be read visually by an embodiment's user, if the application so desires, and/or any method's results may be stored in the one or more memory devices for recordation and later access. Alternatively, the results may be read by someone other than the user or the supplier of the sample. In some circumstances, the results of the method will be restricted from the user of the embodiment and/or the supplier of the sample analyzed.

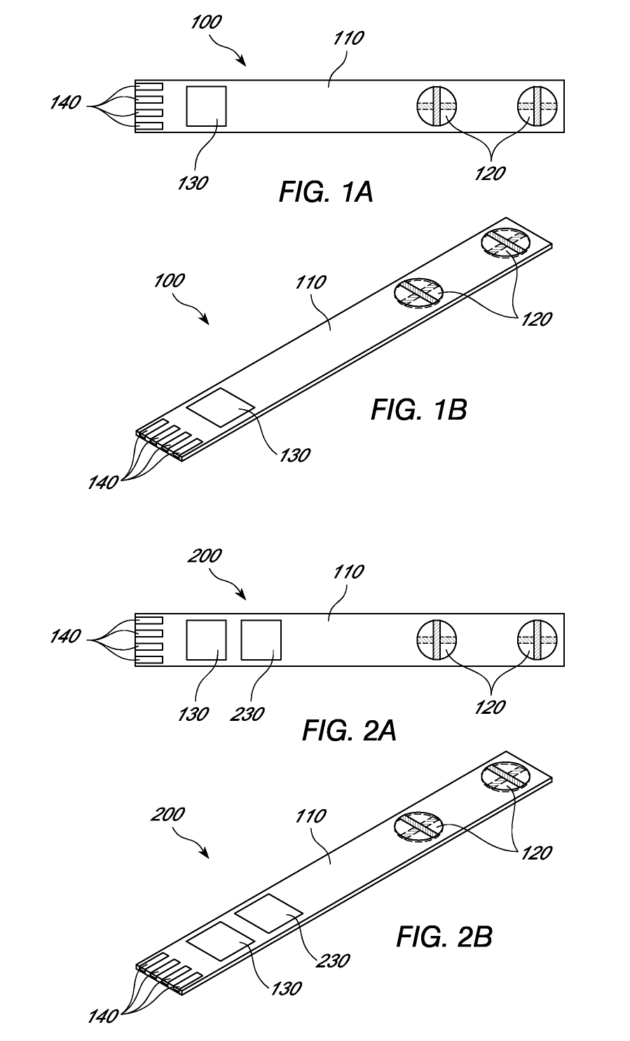

Referring to the drawings, FIG. 1A illustrates schematically a top view of an embodiment of a diagnostic test strip, 100, having two test pads, 120, and a memory device, 130. In some embodiments, memory device 130 is a write once flash memory. Other types of information storage devices may alternatively be used for memory device 130. In some embodiments, memory device 130 comprises multiple types of memory. For example, memory device 130 comprises both a write once memory and a write many memory. Diagnostic test strip 100 has connector pins, 140, which facilitate the transmission of information to and from memory device 130. In this embodiment, memory device 130 and test pads 120 are separated such that test pads 120 are not in fluid communication with memory device 130. The connector pins, 140, are configured to conduct electronic signals to and from memory device 130 via conductors (not shown) on carrier strip 110, which are electrically connected to connector pins 140 and memory device 130. Accordingly, connector pins 140, may be connected to a computer or other device to write information to or access information stored in memory device 130. In some embodiments, memory device 130 is an RF memory, which can communicate wirelessly. In some embodiments, memory device 130 comprises an RFID. Accordingly, in such embodiments connector pins 140 may be omitted. FIG. 1B illustrates schematically a perspective view of diagnostic test strip 100.

FIG. 2A illustrates schematically a top view of an embodiment of a diagnostic test strip, 200, having two test pads, 120, and two memory devices, 130 and 230. In some embodiments, memory device 130 is a write once memory and memory device 230 is a write many memory. Diagnostic test strip 200 has connector pins, 140, which facilitate the transmission of information to or from memory devices 130 and/or 230. Memory devices 130 and 230 are separated from test pads 120 such that test pads 120 are not in fluid communication with memory devices 130 and 230. The connector pins, 140, are configured to conduct electronic signals to and from memory devices 130 and 230 via conductors (not shown) on carrier strip 110, which are electrically connected to connector pins 140 and memory device 130. Accordingly, connector pins 140, may be connected to a computer or other device to write information to or access information stored in memory devices 130 and 230. FIG. 2B illustrates schematically a perspective view of diagnostic test strip 200.

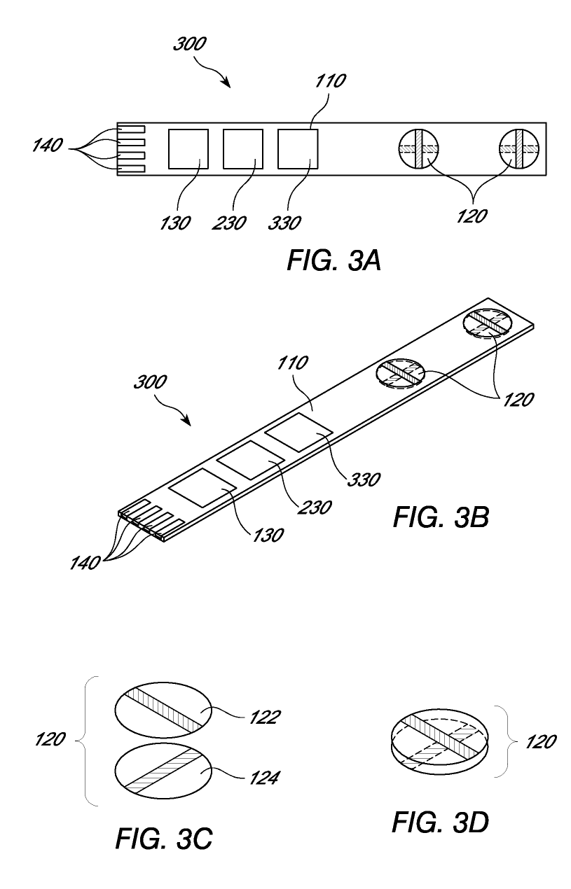

FIG. 3A illustrates schematically a top view of an embodiment of a diagnostic test strip, 300, having two test pads, 120, and two memory devices, 130 and 230, and a network controller 330. In this embodiment, memory device 130 is a write once flash memory, and memory device 230 is a write many flash memory. Diagnostic test strip 300 has connector pins, 140, which facilitate the transmission of information to and from memory devices 130 and 230, and/or network controller 330. Memory devices 130 and 230, and network controller 330 are separated from test pads 120 such that test pads 120 are not in fluid communication with memory devices 130 and 230, and network controller 330. The network controller 330 can be used to manage communication between the memory devices 130 and 230, and a network connected to connector pins 140. In some embodiments, the network controller 330 manages encryption and decryption for secure transmission of the information. The connector pins, 140, are configured to conduct electronic signals to and from memory devices 130 and 230 via conductors (not shown) on carrier strip 110, which are electrically connected to connector pins 140 and memory device 130. Accordingly, connector pins 140, may be connected to a computer or other device to write information to or access information stored in memory devices 130 and 230, using network controller 330. FIG. 3B illustrates schematically the same embodiment illustrated in FIG. 3A in a perspective view, rather than a top view. FIG. 3B illustrates schematically a perspective view of diagnostic test strip 300.

FIG. 3C illustrates test pad 120 as comprising two test pad layers, 122 and 124. Test pad layers 122 and 124 generate signals upon detection of one or more analytes and/or markers of analytes. FIG. 3D further illustrate how layers 122 and 124 can be stacked such that the signals produce a “plus” sign.

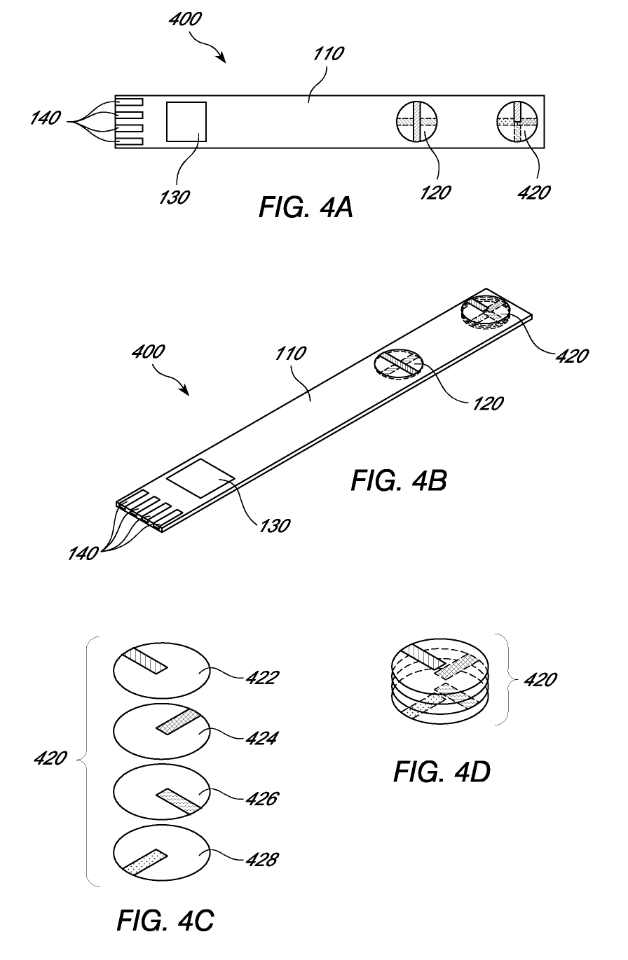

FIG. 4A illustrates schematically a top view of an embodiment of a diagnostic test strip, 400, having two test pads, 120 and 420, and a single memory device, 130. In this embodiment, memory device 130 is a write once flash memory. Diagnostic test strip 400 has connector pins, 140, which facilitate the transmission of information to and from memory device 130. Memory device 130 and test pads 120 and 420 are separated such that test pads 120 and 420 are not in fluid communication with memory device 130. The information stored in memory device 130 can be transferred to a computer, network, or other device capable of accessing information stored in memory device 130. FIG. 4B illustrates schematically a perspective view of diagnostic test strip 400. In FIGS. 4A and 4B, test pad 420 indicates the presence of an analyte, denoted by the “plus” symbol on the test pad. As discussed below, the “plus” symbol may be indicative of both the presence and/or concentration of a particular analyte.

In FIGS. 4A and 4B, test pads 120 and 420 are optionally composed of optically transparent multiple layers, illustrated schematically for test pad 420 in FIGS. 4C and 4D. FIG. 4C schematically illustrates test pad 420 as comprising four test pad layers, 422, 424, 426, and 428. Each of the test pad layers 422, 424, 426, and 428 are impregnated with test reagent and/or signaling reagent such that the presence of a particular analyte, marker of an analyte, and/or marker of different analytes will result in the generation of a visual signal. In FIG. 4C, the visual signal is denoted by a shaded line. Upon detection of the presence of a particular analyte, marker of an analyte, and/or marker of different analytes, in each of the test pad layers 422, 424, 426, and 428, a visual signal is generated in each layer that appears to the observer as a “plus” sign. This “plus” sign may confirm the presence of a single analyte or multiple analytes which may confirm the diagnosis of a disease, illness, or injury. Thus, the use of test pads having layers comprising optically transparent material impregnated with test reagent and/or signaling reagent provides a user with the ability to perform a more complex analysis. FIG. 4D schematically illustrates the stacking of multiple test pad layers to afford a test pad such as 420.

Alternatively, test pads having multiple layers, such as test pad 420, may indicate the concentration of one or more analytes present in a sample. For example, test pad layers 422, 424, 426, and 428 may have different sensitivities to a particular analyte that correlate to a particular concentration of an analyte in a sample. At a certain concentration of analyte, test pad layer 422 may detect the analyte and generate a signal, such as the shaded line indicated in FIGS. 4C and 4D. At a higher concentration of analyte, both test pad layers 422 and 424 may detect the presence of the analyte and generate a signal, such as the shaded lines indicated in FIGS. 4C and 4D. Still yet, at a higher concentration of analyte, test pad layers 422, 424, and 426 may detect the presence of the analyte and generate a signal, such as the shaded lines indicated in FIGS. 4C and 4D. Finally, at an even higher concentration of analyte, all four test pad layers 422, 424, 426, and 428 may detect the presence of the analyte and generate a signal, such as the shaded lines indicated in FIGS. 4A, 4B, 4C, and 4D. Consequently, the different sensitivities of test pad layers 422, 424, 426, and 428 afford information to an observer about the presence and concentration of an analyte in a sample.

One can readily appreciate the application of such an embodiment of a multiple layer test pad when knowledge of a certain concentration is needed. As a non-limiting application, the detection of a person's blood level alcohol may be achieved using such an embodiment. If test pad layer 422 was sensitive to a blood alcohol level of at least 0.02%, test pad layer 424 was sensitive to a blood alcohol level of at least 0.04%, test pad layer 426 was sensitive to a blood alcohol level of at least 0.06%, and test pad layer 428 was sensitive to a blood alcohol level of at least 0.08%, then the application of a sample having a blood alcohol level at least at the sensitive percentages would generate a signal. Assuming that operating a motor vehicle with a blood alcohol level equal to or greater than 0.08% is illegal, then the application of a sample that generates a “plus” sign as in FIGS. 4A-4D would indicate that the sample provider should not operate a motor vehicle.

Diagnostic test strips having memory, such as those discussed above, can be used in various applications. For example, a memory, can be written as part of manufacturing a diagnostic test strip or as part of preparing a diagnostic test strip for sale. The memory can be written with, for example, a unique identification code, with manufacturing information, such as a batch or lot number, a date, a place, and/or information identifying equipment or a manufacturing line. In some embodiments, the unique identification code may additionally be formed on the diagnostic test strip so as to be visible.

In some embodiments, at or near the time of using the diagnostic test strip, information regarding the test being performed may be written to the memory. For example, the information may be entered into a computing device and downloaded to the memory. The information may be related to, for example, circumstances regarding the assay, such as time, date, environmental conditions, identification of the subject tested, and identification of the individual and administering the test. In some embodiments, the information may relate to the subject tested. For example, the information may relate to identification, demographic data, such as gender and age, physiological data such as temperature and blood pressure. In some embodiments, the information may relate to observations of the test administrator, such as an assessment of mental state of the test subject, or a description of the activities of the test subject. In some embodiments, the information may relate to results of the test. For example, a camera or other scanning device can be used to record the results indicated on the test pads. The picture or other record of the diagnostic test strip is downloaded to the diagnostic test strip. In some embodiments, the picture or other record of the diagnostic test strip is stored in a database and is associated with information in the memory of the diagnostic test strip.

### Memory Devices

Memory devices suitable for inclusion in embodiments are well known in the art. In some embodiments, one or more flash memory devices are incorporated. The flash memory devices may be single-level cell devices or multi-level cell devices. The memory cells comprising the flash memory device my include conducting floating gates or non-conductive floating gates. The flash memory devices may be of a NOR flash device, a NAND flash device, or a serial flash device. The flash memory devices may optionally store information including, but not limited to manufacture date, manufacturer, testing date, testing time, testing location, the presence and/or absence of any analyte upon testing, the concentration of any analyte, and/or indentifying characteristics of any test reagents contained on the one or more test pads. Flash memory devices may optionally be read-only devices, such as write once flash memory, or they may optionally be rewritable. When write once flash memory is used, the embodiments are particularly suited for legal applications because courts have acknowledged that such devices are unalterable and admissible as evidence. Such devices are also particularly useful when a chain of evidence needs to be established.

In one embodiment, the one or more memory devices may optionally include combinations of read-only devices and rewritable devices. The one or more memory devices may optionally be in communication with a device capable of communicating with a network. In some embodiments, the one or more memory devices communicate with a computer network wirelessly. In other embodiments, the one or more memory devices may communicate with a computer or network with a hard line. Information stored in the one or more memory devices, or optionally communicated to a computer network, may be encrypted. Such encryption is well known in the art and any known method of encrypting information may be used in an embodiment. Examples of encryption include, but are not limited to, a symmetrical encryption key or a public/private key where the encryption key is optionally provided the user of an embodiment or a third person interested in the encrypted information.

### Connecter Pins

Connector pins suitable for inclusion in an embodiment are well known in the art. Such connector pins include, but are not limited to, bus connector pins, USB data bus pins, serial data bus pins, parallel data bus pins, 1394 data bus pins, network connector pins, and Ethernet connections.

### Carrier Strip

The carrier strip facilitates the transmission of information from the one or more test pads to the one or more memory devices. Transmitted information may include, but is not limited to, the presence, absence, and/or concentration of one or more analytes of interest. The carrier strip may facilitate the transmission of information from the one or more test pads to the one or more memory devices by any of several methods known in the art. Such methods include, but are not limited to, the transmission of electrical signals which result from changes in the coulometry, amperometry, or potentiometry of the materials comprising the carrier strip. See U.S. Pat. No. 6,743,635 (Neel et al., issued on Jun. 1, 2001) and U.S. Pat. No. 6,946,299 (Neel at al., issued on Sep. 20, 2005), which are herein incorporated by reference. Alternatively, the carrier strip may facilitate the transmission of optical signals which result from differences in the reflection, transmission, scattering, absorption, fluorescence, or electrochemiluminescense of the materials comprising the carrier strip and/or the test pads. See U.S. Pat. No. 6,040,195 (Carroll et al., issued on Mar. 21, 2000) and U.S. Pat. No. 6,284, 550 (Carroll et al., issued on Sep. 4, 2001) which are herein incorporated by reference.

Additionally, the carrier strip provides structural support for the one or more test pads and the one or more boundary projections. As a structural support, many materials suitable for use in preparing the carrier strip are known in the art. Such materials include but are not limited to plastics including polyethylene terephthalate, high-density polyethylene, polypropylene, cellulose, Bakelite, polystyrene, high impact polystyrene, acrylonitrile butadiene styrene, polyester, polyurethanes, polycarbonates, polycarbonate/acrylonitrile butadiene styrene, polymethyl methacrylate, polytetrafluoroethylene, polyetherimide, phenol formaldehydes, urea-formaldehyde, melamine formaldehyde, polylactic acid, plastarch material, polyvinylchloride, nylon, and other polyamides, metals, alloys, ceramics, glass, wood, cardboard, paper, natural rubber, synthetic rubber, and other suitable polymers. Optionally, the carrier strip may be porous or non-porous.

The carrier strip's size and shape is only limited by the desired application of the embodiment. For example, if the desired application is testing a human patient, the embodiment, and consequently the carrier strip, may be smaller or larger depending upon the size of the human patient Likewise, if the desired application involves testing an animal patient, the embodiment, and consequently the carrier strip, may be smaller or larger depending upon the size of the animal patient. In some embodiments, the carrier strip is about 1, about 1.25, about 1.5, about 1.75, about 2, about 2.25, about 2.5, about 2.75, about 3, about 3.25, about 3.5, about 3.75, about 4, about 1-2, about 1-3, about 1-4, about 2-3, about 2-4, or about 3-4 inches in length. The carrier strip's shape may optionally be varied depending upon the desired application of the embodiment. Some applications may require substantially narrow, fat, rectangular, circular, oval, square, triangular, or other shapes, including combinations of the indicated shapes. It is envisioned that the shape of embodiments can be tailored to the shape of the environment in which the embodiments will be applied. Moreover, the carrier strip may contain boundary projections that substantially surround one, two, three, and/or four sides of one or more test pads to collect and/or direct sample application to the one or more test pads. Furthermore, it is envisioned that a handle may be optionally attached to a carrier strip or in contact with a carrier strip, either directly or indirectly.

### Test Reagents and Signaling Reagents

Test reagents and signaling reagents suitable for inclusion in embodiments are well known in the art. Such reagents include, but are not limited to, polyclonal antisera and monoclonal antibodies that have specific binding properties and high affinity for virtually any antigenic substance. Literature affords many means of preparing such reagents. See, e.g., Laboratory Techniques in Biochemistry and Molecular Biology, Tijssen, Vol. 15, Practice and Theory of Enzyme Immunoassays, chapter 13, The immobilization of Immunoreactants on Solid Phases, pp. 297-328, and the references cited therein which are herein incorporated by reference. Additional assay protocols, reagents, and analytes useful in the practice of the invention are known per se. See, e.g., U.S. Pat. No. 4,313,734 (Leuvering, issued on Feb. 2, 1982), columns 4-18, and U.S. Pat. No. 4,366,241(Tom et al., issued on Dec. 28, 1982), columns 5-40 which are herein incorporated by reference.

Metal sols, including but not limited to gold sol, and other types of colored particles, including but not limited to, organic dye sols and colored latex particles, that are useful as marker substances in immunoassay procedures are also known per se and suitable for use as test reagents and/or signaling reagents. See, for example, U.S. Pat. No. 4,313,734 (Leuvering, issued on Feb. 2, 1982), the disclosure of which is incorporated herein by reference. For details and engineering principles involved in the synthesis of colored particle conjugates see Horisberger, Evaluation of Colloidal Gold as a Cytochromic Marker for Transmission and Scanning Electron Microscopy, Biol. Cellulaire, 36, 253-258 (1979); Leuvering et al, Sol Particle Immunoassay, J. Immunoassay 1 (1), 77-91 (1980), and Frens, Controlled Nucleation for the Regulation of the Particle Size in Monodisperse Gold Suspensions, Nature, Physical Science, 241, pp. 20-22 (1973) which are herein incorporated by reference.

Test reagents for inclusion in the embodiments may signal directly, such as with an electrical or optical signal (visible either to the naked eye, or with an optical filter or upon applied stimulation to promote fluorescence or phosphorescence). Test reagents may also signal indirectly such as with enzymes, e.g. alkaline phosphatase and/or horseradish peroxidase, in combination with signaling reagents in the form of enzymatic substrates that will generate a signal upon interaction with the enzyme. In some embodiments, the signaling reagent and/or test reagent is incorporated into the test pad. In other embodiments, the signaling reagent and/or test reagent is added to the test sample before application to the test pad. In additional embodiments, the signaling reagent and/or test reagent is added to the test pad after introduction of the test sample.

Alcohol sensitive test reagents and methods are well known in the art. See, e.g. U.S. Pat. No. 5,563,073 (Titmas, issued on Oct. 8, 1996) and Jai Moo Shin et al., Simple Diagnostic Tests to Detect Toxic Alcohol Intoxications, NIH (October 2008), which are hereby incorporated by reference in their entirety. In some embodiments, the test reagent and/or signaling reagent from Alco Screen™ pads, manufactured by Chematics, Inc. located in North Webster, Ind., is incorporated. Optionally, the test reagent and/or signaling reagent from Alco Screen™ pads is incorporated in the one or more test pads, but it may also be applied to the test pad after sample application or it may be applied to the sample before application to the test pad. In some embodiments the test reagent and/or signaling reagent from the alcohol dehydrogenase method (ADH method) is incorporated in the one or more test pads, but it may also be applied to the test pad after sample application or it may be applied to the sample before application to the test pad. In some embodiments the test reagent and/or signaling reagent from the alcohol oxidase method method (ALOx method) is incorporated in the one or more test pads, but it may also be applied to the test pad after sample application or it may be applied to the sample before application to the test pad. In some embodiments the test reagent and/or signaling reagent from the sodium periodate method is incorporated in the one or more test pads, but it may also be applied to the test pad after sample application or it may be applied to the sample before application to the test pad. In some embodiments the test reagent and/or signaling reagent from the potassium permanganate method (PA method) is incorporated in the one or more test pads, but it may also be applied to the test pad after sample application or it may be applied to the sample before application to the test pad.

Test reagents and/or signaling reagents may also detect the storage and handling of embodiments. In some embodiments, test reagents and/or signaling reagents may be sensitive to temperature and if the temperature of the embodiment's environment has exceeded or fallen below a predetermined temperature, optionally for a predetermined period of time, the test reagents and/or signaling reagents may be inactivated. Optionally, the inactivation of the test reagents and/or signaling reagents may result in the transmission of a signal to the one or more memory devices and/or to the user of the embodiment.

In some embodiments, test reagents and/or signaling reagents may be sensitive to moisture, and if the humidity of the embodiment's environment has exceeded or fallen below a predetermined level, optionally for a predetermined period of time, the test reagents and/or signaling reagents may be inactivated. Optionally, the inactivation of the test reagents and/or signaling reagents may result in the transmission of a signal to the one or more memory devices and/or to the user of the embodiment.

Test reagents and/or signaling reagents may also detect whether a sufficient amount of sample has been applied to an embodiment for analysis. For example, when the sample is saliva, a test reagent and/or signaling reagent specific for a salivary enzyme, such as amylase, may detect the salivary enzyme's presence if a sufficient volume of sample has been applied. The detection of a sufficient sample may optionally be signaled to the user in the form of a color or symbol. Using such embodiments, the user would then know if a sufficient quantity of sample was applied to the one or more test pads to afford an accurate analysis.

Embodiments that detect storage and/or sufficient application of sample volume are particularly capable of reducing the occurrence of false negatives. For example, poor storage conditions may inactivate a test reagent in a test pad. Upon application of sample to such a test pad, no signal may result and a user could believe that an analyte is not present—a false negative. Alternatively, test pads having a pre-printed negative signal may suffer a similar occurrence of a false negative if the test reagent is inactivated because an analytes presence in a sample would not convert the pre-printed negative signal into a positive signal. Likewise, an insufficient volume of sample may generate no signal or a negative signal and cause a user to believe that an analyte is not present.

Any enzyme, antibody, dye buffer, chemical, sol, or combinations thereof may be incorporated so long as the enzyme, antibody, dye buffer, chemical, metal sol, or combinations thereof are capable of detecting the presence of one or more analytes in a sample. See, e.g., U.S. Pat. No. 6,383,736 (Titmas, issued on May 7, 2002), U.S. Pat. No. 7,858,756 (Owens et al., issued on Dec. 28, 2010), and U.S. Pat. No. 7,790,400 (Jehanli et al., issued on Sep. 7, 2010) which are hereby incorporated by reference in their entirety.

### Test Pads

The one or more test pads may be prepared from any bibulous, porous, fibrous, or sorbent material capable of rapidly absorbing a sample. Porous plastics material, such as polypropylene, polyethylene, polyvinylidene flouride, ethylene vinylacetate, acrylonitrile and polytetrafluoroethylene can be used. Optionally, the one or more test pads can be pre-treated with a surface-active agent to reduce any inherent hydrophobicity in the one or more test pads and enhance their ability to absorb a sample. Moreover any one of the one or more test pads may be treated with an oxygen-impermeable water soluble substance. Suitable examples of an oxygen-impermeable water soluble substance include, but are not limited to, polyvinyl alcohol, partly saponified polyvinyl acetate which can also contain vinylether and vinylacetal units, polyvinyl pyrrolidone and copolymers thereof with vinyl acetate and vinyl ethers, hydroxy alkyl cellulose, gelatin, polyacrylic acid, gum arabic, polyacryl amide, dextrin, cyclodextrin, copolymers of alkylvinyl ethers and maleic acid anhydride, ring opened polymers of maleic acid anhydride, water-soluble high molecular polymers of ethylene oxide having molecular weights of above 5,000, and/or polyvinyl alcohol in combination with poly(1-vinylimidazole) or a copolymer of 1-vinyl-imidazole. The one or more test pads can also be made from paper or other cellulosic materials, including but not limited to nitrocellulose. Materials that are now used in the nibs of fiber-tipped pens are also suitable for incorporation in the one or more test pads.

Optionally, the one or more test pads may be prepared from non-porous materials. In such circumstances, the test reagents and/or signaling reagents may be coated on the outer surface of the one or more test pads such that contact with a sample containing an analyte will result in the generation of a signal.

Using known methods, test pads may be shaped or extruded in a variety of lengths and cross-sections. Embodiments may possess one or more test pads of various sizes and shapes, and the size and shape of the one or more test pads are only limited by their number, size, and desired application of the embodiment in which they are incorporated within. In some embodiments, the one or more test pads are substantially similar in size and/or shape. In other embodiments, the one or more test pads may differ substantially in size and/or shape. It is readily envisioned that embodiments may possess about one or more test pads, about two or more test pads, about three or more test pads, about four or more test pads, about five or more test pads, about six or more test pads, about seven or more test pads, about eight or more test pads, about nine or more test pads, about ten or more test pads, about 1-4 test pads, about 1-10 test pads about 1-100 test pads, about 2-100 test pads, about 3-100 test pads, about 4-100 test pads, about 5-100 test pads, about 5-75 test pads, about 10-50 test pads, about 15-25 test pads, and individual numbers of test pads therein. The one or more test pads may be made of the same material, or optionally they may be made of different materials or even combinations of different materials. Moreover, the one or more test pads may be recessed into the carrier strip.

In some embodiments, test pads may be prepared from a single layer of material. In other embodiments, test pads may be prepared from multiple layers of material. It is readily envisioned that embodiments may possess about one or more layers, about two or more layers, about three or more layers, about four or more layers, about five or more layers, about six or more layers, about seven or more layers, about eight or more layers, about nine or more layers, about ten or more layers, about 1-4 layers, about 1-5 layers, about 1-6 layers, about 1-7 layers, about 1-8 layers, about 1-9 layers, about 1-10 layers, about 1-100 layers, about 2-100 layers, about 3-100 layers, about 4-100 layers, about 5-100 layers, about 5-75 layers, about 10-50 layers, about 15-25 layers, and individual numbers of layers therein.

The test pad layers may be of the same or different materials. Test reagents and/or signaling reagents may also be impregnated in a single layer of material or in multiple layers of material. The impregnation may take any suitable form, including, but not limited to, a substantially uniform impregnation or impregnation with dots or stripes. Test reagents and/or signaling reagents can be impregnated in various concentrations in one or more of the multiple layers to tailor the sensitivity of the test pads to certain analytes. Such sensitivity could afford information about the concentration of an analyte in the sample. Furthermore, the impregnation may optionally be conducted in a manner that will generate a signal observable by the user upon application of a sufficient quantity of sample, detection of an analyte, or proper/improper storage of the embodiment.

When one or more test pads are comprised of multiple layers of material, one or more layers of material may be impregnated (e.g. pre-printed) with an inert chemical such that a line or “minus sign” is displayed to the user. In some embodiments, the line or “minus sign” could be in the form of a material covering the one or more test pads to give a visual impression of a line or “minus sign” on the one or more test pads. One or more additional layers of the material comprising the one or more test pads could then be impregnated with a test reagent and/or a signaling reagent that upon detecting a sufficient quantity of sample, appropriate storage temperature, and/or the presence of an analyte, the impregnated test reagent and/or signaling reagent will create a perpendicular line such that a “plus sign” will be signaled to the user. In other embodiments, the line or “minus sign” displayed in the one or more test pads could be obscured by color or opaqueness when a test reagent and/or a signaling reagent detects a sufficient quantity of sample, appropriate or inappropriate storage temperature, and/or the presence of an analyte.

The test pad layers may comprise optically transparent membranes. Detection on an analyte may then generate a signal that is opaque, partially transparent, or completely transparent. Moreover, test pad layers may be only partially optically transparent prior to application of a sample. Alternatively, the application of a sample to one or more test pad layers may result in the layers becoming optically transparent, thereby allowing a user to see generated and/or pre-printed signals on test pad layers below the optically transparent layers. Moreover, the individual layers in a test pad may be positioned such that the detection of an analyte in a lower layer of material is obscured by the detection of an analyte in a layer of material positioned above the lower layer.

It is also envisioned that embodiments may have arrangements of test pads and/or arrangements of layers within multiple layered test pads such that the detection of an analyte in the test pads or the layers of a test pad generate a signal, such as a “plus sign” or “minus sign” to the user. Such embodiments may comprise at least two layers of material, each capable of generating a line upon detecting an analyte or a certain concentration of an analyte. Optionally, the lines may intersect to generate a “plus” sign or other signal upon the detection of an analyte in the at least two layers of material. Alternatively, embodiments may comprise at least four layers of material, each capable of generating a line upon detecting an analyte or a certain concentration of an analyte in the at least four layers of material. Optionally, the lines may intersect at one or more points such that a “plus” sign or other symbol is formed. While the aforementioned embodiments have been discussed with reference to “minus” and “plus” signs, it is envisioned that any symbol, including color changes, could be used to convey similar information to a user. Such symbols include, but are not limited to, circles, ovals, squares, triangles, trapezoids, rhombi, plus signs, minus signs, “X” shaped signs, checkmarks, and/or dotted, dashed, or differentially colored version of said symbols. The meaning of any desired symbol or color change could be included in the packaging of an embodiment or imprinted on an embodiment.

The test reagents applied to each layer of material may optionally be the same or different. When different test reagents are applied to different layers of material comprising the one or more test pads, the test pad may be tailored to generate a signal indicating the diagnosis of one or more illnesses, diseases, or injuries. One method for achieving such a diagnosis would be to have the individual layers comprising the test pad generate a signal in response to one or more symptoms of one or more illnesses, diseases, or injuries. For example, if the diagnosis of one or more illnesses, diseases, or injuries required the determination of multiple analytes, then the detection of each analyte could produce a portion of a symbol that is visible to the user. Upon formation of a complete symbol, the embodiment would confirm the presence of a certain illness, disease, or injury. Optionally, information relating to each specific analyte could be transferred to the one or more memory devices.

One can readily appreciate the application of such embodiments of multiple layer test pads when knowledge of a certain concentration is needed. As a non-limiting application, the detection of a person's blood alcohol level may be achieved using such an embodiment. For a test pad comprising at least four test pad layers, if a first test pad layer was sensitive to a blood alcohol level of at least 0.02%, a second test pad layer was sensitive to a blood alcohol level of at least 0.04%, a third test pad layer was sensitive to a blood alcohol level of at least 0.06%, and a fourth test pad layer was sensitive to a blood alcohol level of at least 0.08%, then the application of a sample having a blood alcohol level at least at the sensitive percentages would generate a signal. Assuming that operating a motor vehicle with a blood alcohol level equal to or greater than 0.08% is illegal, then the application of a sample that generates a “plus” sign would indicate that the sample provider should not legally operate a motor vehicle. One will readily appreciate that this described example is capable of extension to any number of test pads having any number of layers, such that the detection of an analyte in each layer generates a signal indicative of concentration.

As another non-limiting example, test reagents and/or signaling reagents that are sensitive to markers specific for hepatitis and/or liver damage may be applied to test pads and/or layers within test pads. Consequently, the detection of markers specific for hepatitis and/or liver damage in each test pad and/or layers within test pads would generate a signal. An individual test pad may optionally be sensitive to a single marker for hepatitis and/or liver damage. Alternatively, a single test pad may be sensitive to multiple markers for hepatitis and/or liver damage. In such an embodiment, the detection of one or more markers for hepatitis and/or liver damage may produce a certain signal, e.g. color, indicative of the number of markers detected and/or indicative of the exact marker detected. Alternatively, an embodiment may produce a signal in the form of a shape that indicates the presence of one or more markers indicative of hepatitis and/or liver damage. For example, an embodiment may have a test pad with four or more test pad layers, while each layer may be sensitive to one or more markers specific to an analyte such as viral hepatitis. The respective detection of a marker in each of the test pad would generate a signal such that the detection of a marker in each of the test pad layers would confirm the diagnosis of a viral hepatitis. Although such an embodiment has been described with specific references to a viral hepatitis, it is envisioned that such an embodiment may readily be tailored to detect any number of analytes and/or markers that are specific to any analyte described below.

Embodiments may optionally possess one or more test pads and test reagents that detect analytes important to a certain age population (e.g. infants, children, young adults, adults, or elderly individuals). It is also envisioned that embodiments could possess one or more test pads and test reagents that detect analytes important to certain categories of individuals (e.g., law enforcement agents, government employers, military members, chronic drug users, physicians, veterinarians, dentists, parents, private sector employers, aid workers, inmates, hospital patients, nursing home patients, outdoorsmen, immuno-compromised individuals, or students). Embodiments may also be directed to analytes important to geographic regions (e.g. third-world countries, developed countries, or specific climate regions). Such embodiments of the invention simplify the number of different embodiments that a user must purchase or travel with because users can select embodiments that will detect the analytes the users are most interested in, or are most pertinent to a user's current or impending circumstances.

In one embodiment, a single test pad contains or has applied to it a single test reagent and/or signaling reagent suitable for detecting a single analyte. In another embodiment, two or more test pads contain or have applied to one or more of them a single test reagent and/or signaling reagent suitable for detecting a single analyte. Optionally, the single test reagent and/or signaling reagent on or applied to the two or more test pads may be the same or different. Furthermore, when different test reagents and/or signaling reagents are used, the test reagents may be sensitive to the same marker on an analyte or the test reagents may be sensitive to different markers on an analyte. The analyte may optionally be the same or different. When different analytes and different test reagents and/or signaling reagents are used, the analytes and test reagent and/or signaling reagents may be tailored to detect different symptoms of the same illness, disease, or injury. In some embodiments, a diagnosis can be made based upon the detection of all the symptoms specific to an illness, disease, or injury. In other embodiments, a diagnosis can be made based upon the absence of one or more analytes specific to an illness, disease, or injury. Using these described test pads, it is readily apparent that the reduction of false negatives and false positives can be achieved by including redundancy in the embodiments.

In one embodiment, a single test pad may contain or have applied to it two or more reagents suitable for detecting and/or signaling a single analyte. These two or more test reagents and/or signaling reagents may be sensitive to the same marker of an analyte. Optionally, these two or more reagents may be sensitive to different markers on the same analyte. In some embodiments, the two or more test reagents and/or signaling reagents may be applied to the same region of the test pad. In other embodiments, the two or more test reagents and/or signaling reagents may be applied to different regions of the same test pad. The number of test reagents and/or signaling reagents suitable for incorporation or application to a single test pad is limited only by the application of the diagnostic test strip. It is readily envisioned that embodiments may possess about one or more, about two or more, about three or more, about four or more, about five or more, about six or more, about seven or more, about eight or more, about nine or more, about ten or more, about 1-4, about 1-10, about 1-100, about 2-100, about 3-100, about 4-100, about 5-100, about 5-75, about 10-50, about 15-25, and individual numbers therein, of test reagents and/or signaling reagents incorporated or applied to one or more test pads. Using these described test pads, it is readily apparent that the reduction of false negatives and false positives can be achieved by including redundancy in the embodiments.

The one or more test pads suitable for use in an embodiment will readily detect analytes present in liquid samples, such as saliva. It is also envisioned that a test pad may be capable of detecting an analyte present in solid and/or semi-solid samples. When solid and/or semi-solid samples are analyzed, it is understood that a liquid may optionally be applied to the test pad to facilitate analysis.

When liquids and/or liquid samples are applied to test pads, lateral flow through material may result from surface tension, cohesion, adhesion, wicking, and/or capillary action. In general, embodiments that utilize lateral flow will require substantial amounts of a liquid sample for sufficient contacting of the sample with a devices test area. In some embodiments, lateral flow is confined to the test pad region. In other embodiments, lateral flow is confined to individual test pads. In further embodiments, lateral flow is confined to individual layers of a multi-layer test pad. Moreover, some embodiments overcome the use of lateral flow by having a test pad designed to absorb the fluid sample without requiring surface tension, cohesion, adhesion, wicking, and/or capillary action to contact the fluid sample with the test area. Such embodiments are particularly suited for use when the volume of a fluid sample is small and/or limited. This includes, but is not limited to, instances when the fluid sample is oral fluid such as saliva.

### Analytes

An assay based on the principles described herein can be used to determine a wide variety of analytes by choice of appropriate test reagents and/or signaling reagents. The embodiments described herein can be used to test for the existence of analytes including, but not limited to, drugs, especially drugs of abuse; heavy metals; pesticides; pollutants; proteins; polynucleotides such as DNA, RNA, rRNA, tRNA, mRNA, and siRNA; hormones; vitamins; microorganisms such as bacteria, fungi, algae, protozoa, multi-cellular parasites, and viruses; tumor markers; liver function markers; kidney function markers; blood coagulation factors; and toxins. The embodiments may also optionally detect metabolites of each of the aforementioned examples of analytes. Furthermore, some embodiments may also detect their storage conditions, specifically the temperature and humidity of their environment, and/or the application of an appropriate quantity of sample for analysis.

Analytes may be reference analytes or target analytes. Any given analyte may be either a reference analyte or a target analyte, depending upon the desired application. Indeed, any analyte described below that is known to consistently be present in a given sample may serve as a reference analyte. As a non-limiting example, alpha-amylase is an enzyme present in saliva and could serve as a reference analyte when the analyzed sample is saliva. However, methadone could serve as a reference analyte when an embodiment is desired for use with samples obtained from patients generally known and/or suspected of having methadone in their system. Thus, one will readily appreciate that it is the application of the embodiment that determines the analytes classified as references or targets.

More specific examples of drug analytes, including both drugs of abuse and therapeutic drugs, include opiates, which includes but is not limited to methadone, morphine, heroin, dextromethorphen, meperidine, codeine, hydromorphone, pholcodine, and metabolites thereof.

More specific examples of drug analytes, including both drugs of abuse and therapeutic drugs, include benzheterocyclics, the heterocyclic rings being azepines, diazepines and phenothiazines. Examples of azepines include fenoldopam. Examples of benzodiazepines include alprazolam, bretazenil, bromazepam, chlorodiazepoxide, cinolazepam, clonazepam, cloxazolam, clorazepate, diazepam, estazolam, fludiazepam, flunirazepam, flurazepam, flutoprazepam, halazepam, ketazolam, loprazolam, lorazepam, lormetazepam, medazepam, midazolam, nimetazepam, nitrazepam, nordiazepam, N-Desmethyld, oxazepam, phenazepam, pinazepam, prazepam, premazepam, quazepam, temazepam, tetrazepam, triazolam, and other benzodiazepine receptor ligands such as clobazam, DMCM, flumazenil, eszopiclone, zaleplon, zolpidem, and zopiclone. Examples of phenothiazines include chlorpromazine, promethazine, triflupromazine, methotrimeprazine, mesoridazine, thioridazine, fluphenazine, perphenazine, prochlorperazine, and trifluoperazine. Examples of other benzheterocyclics include, but are not limited to, carbamazepine and imipramine.

Additional drug analytes, including both drugs of abuse and therapeutic drugs, include alkaloids, such as agents that interact with opioid receptors including morphine, dihydromorphine, desomorphine, hydromorphone, nicomorphine, oxymorphone, hydromorphinol, nalbuphine, naloxone, naltrexone, buprenorphine, etorphine, metopon, diacetyldihydromorphine, thebacon, methodone, codeine, hydrocodone, dihydrocodeine, oxycodone, papaveretum, oripavine, thebaine, tapentadol, and heroin; agents that exert effects on serotonin receptors, such as cocaine (and other reuptake inhibitors, including norepinephrine, dopamine, and serotonin reuptake inhibitors); cocaine metabolites such as benzoylecgonine; ergot alkaloids; steroid alkaloids; iminazoyl alkaloids; quinazoline alkaloids; isoquinoline alkaloids; quinoline alkaloids; and diterpene alkaloids.

Another group of drug analytes, including both drugs of abuse and therapeutic drugs, includes steroids, including the estrogens, gestogens, androgens, andrenocortical steroids, bile acids, cardiotonic glycosides and aglycones, which includes digoxin and digoxigenin, saponins and sapogenins, their derivatives and metabolites.

Additional drug analytes, including both drugs of abuse and therapeutic drugs, is the barbiturates, such as barbital, allobarbital, amobarbital, aprobarbital, alphenal, brallobarbital, hexobarbital, Phenobarbital, phencyclidine (PCP), pentobarbital, Nembutal, secobarbital, diphenylhydantonin, primidone, and ethosuximide. Additionally, drugs similar in effect to barbiturates are potential analytes, such as methaqualone, cloroqualone, diproqualone, etaqualone, mebroqualone, mecloqualone, methylmethaqualone, and nitromethaqualone.

Another group of drug analytes, including both drugs of abuse and therapeutic drugs, is aminoalkylbenzenes, including the phenethylamines such as amphetamine, methamphetamine, ephedrine, amphepramone, prolintane, lisdexamfetamine, mescaline, and catecholamines, which includes ephedrine, L-dopa, epinephrine, narceine, and papaverine.

Additional drug analytes, including both drugs of abuse and therapeutic drugs, includes those derived from marijuana, which includes cannabinol, tetrahydrocannabinol, 11-nor-9-carboxy-delta-9-tetrahydrocannabinol (THC), nabilone, dronabinol, marinol, and cannabinoids such as cannabidiol, cannabinol, and tetrahydrocannabivarin.

Another group of drug analytes, including both drugs of abuse and therapeutic drugs, are those that interact with the N-methyl d-aspartate (“NMDA”) receptor, including agonists, modulators, and antagonists such as 1-(1-phylcyclohexyl)piperidine (phencyclidine or “PCP”), R-2-amino-5-phosphonopentanoate, 2-amino-7-phosphonoheptanoic acid, (3-[(R)-2-carboxypiperazin-4-yl]-prop-2-enyl-1-phosphonic acid), PEAQX, selfotel, amantadine, dextrallorphan, dextromethorphan, dextrorphan, dizocilpine, ethanol, eticyclidine, gacyclidine, ibogaine, ketamine, memantine, methoxetamine, rolicyclidine, tenocyclidine, tiletamine, neramexane, eliprodil, etoxadrol, dexoxadrol, NEFA, remacemide, delucemine, 8A-PDHQ, aptiganel, HU-211, remacemide, atomoxetine, rhynchophylline, 1-aminocyclopropanecarboxylic acid, 7-chlorokynurenate, 5,7-dichlorokynurenic acid, kynurenic acid, and lacosamide.

Another group of therapeutic drugs is antibiotics, which include, for example, beta-lactam antiobiotics such as penicillins and cephalosporins, penems and carbapenems, antimicrobials such as aminoglycosides, ansamycins, carbacephems, glycopeptides, lincosamides, lipopetides, macrolides, monobactams, nitrofurans, quionolones, polypeptide-based antibiotics, chloromycetin, actinomycetin, spectinomycin, sulphonamides, trimethoprim, tetracyclines, and beta-lactamase inhibitors such as calvulanic acid, tazobactam, and sulbactam.

Other individual miscellaneous drug analytes, including both drugs of abuse and therapeutic drugs, include nicotine, caffeine, cotinine, gamma-hydroxybutyric acid, dextromoramide, ketobemidone, piritramide, dipipanone, phenadoxone, benzylmorphine, codeine, nicocodeine, dihydrocodeinone enol acetate, tilidine, meptazinol, propiram, acetyldihydrocodeine, pholcodine, 3,4-methylenedioxymethamphetamine, psilocybin, 5-methoxy-N,N-diisopropyltryptamine, peyote, 2,5-dimethoxy-4-methylamphetamine, 2C-T-7 (a psychotropic entheogen), 2C-B, cathinone, alpha-methyltryptamine, bufotenin, benzylpiperazine, methylphenidate, dexmethylphenidate, laudanum, fentanyl, mixed amphetamine salts (i.e. Adderall), lisdexamfetamine, dextroamphetamine, dextromethamphetamine, phentermine, phylpropanolamine, ephedrine, pethidine, anabolic steroids, talbutal, butalbital, buprenorphine, xyrem, paregoric, modafinil, difenoxin, diphenoxylate, promethazine, pregabaline, pyrovalerone, atropine, and other Schedule I-V classified drugs, glucose, cholesterol, bile acids, fructosamine, carbohydrates, metals which includes, but is not limited to lead and arsenic, alcohols (i.e. methanol, ethanol, propanol, butanol, ethylene glycol, diethylene glycol, C₅₋₁₀ containing alcohols, and metabolites thereof), meprobamate, serotonin, meperidine, amitriptyline, nortriptyline, lidocaine, procaineamide, acetylprocainearnide, propranolol, griseofulvin, valproic acid, butyrophenones, antihistamines, and anticholinergic drugs, such as atropine.

Pesticide analytes of interest include categories such as algicides, avicides, bactericides, fungicides, herbicides, insecticides, miticides, molluscicides, nematicides, rodenticides, virucides, and specifically polyhalogenated biphenyls, phosphate esters, thiophosphates, carbamates, and polyhalogenated sulfenamides.

Additional chemical analytes of interest include fertilizers such as ammonium derivatives, nitrates, and phosphates; heavy metals such as lead, mercury, uranium, plutonium, arsenic, cadmium, chromium, and nickel

More specific examples of protein analytes include antibodies, protamines, histones, albumins, globulins, scleroproteins, phosphoproteins, mucoproteins, chromoproteins, lipoproteins, nucleoproteins, glycoproteins, proteoglycans, and unclassified proteins, such as somatotropin, prolactin, insulin, and pepsin. A number of proteins found in the human plasma are important clinically and include prealbumin, albumin, α₁-lipoprotein, α₁-acid glycoprotein, α₁-antitrypsin, α₁-glycoprotein, transcortin, 4.6S -postalbumin, tryptophan-poor, α₁-glycoprotein, α₁X-glycoprotein, thyroxin-binding globulin, inter-α-trypsin-inhibitor, Gc-globulin (Gc I-I, Gc 2-1, Gc 2-2), haptoglobin, ceruloplasmin, cholinesterase, α₂-lipoprotein(s), myoglobin, C-reactive Protein, α₂-macroglobulin, α₂-HS-glycoprotein, Zn-α₂-glycoprotein, α₂-neuramino-glycoprotein, erythropoietin, β-lipoprotein, transferrin, hemopexin, fibrinogen, plasminogen, β₂-glycoprotein I, β₂-glycoprotein II, immunoglobulins A, D, E, G, M, prothrombin, thrombin, and protein markers in cancers including, but not limited to, breast cancer, prostate cancer, melanoma, carcinoma, pancreatic cancer, liver cancer, and brain cancer.

Additional protein analytes of interest include alanine aminotransferase and aspartate aminotransferase. Alanine aminotransferase is markedly elevated when hepatitis is present in the liver. Such elevation for alanine aminotransferase may include at least about 1.25, 1.5, 1.75, 2, 2.25, 2.5, 2.75, and 3.0 times the normal levels associated with a person lacking liver damage. Aspartate aminotransferase is elevated when cellular damage occurs, such as liver damage, skeletal muscle damage, and acute myocardial infarction. Additionally, levels are elevated because of congestive heart failure, pericarditis, cirrhosis, metastatic liver disease, skeletal muscle diseases, and generalized infections such as mononucleosis. Such elevation for aspartate aminotransferase may include at least about 1.25, 1.5, 1.75, 2, 2.25, 2.5, 2.75, and 3.0 times the normal levels associated with a person lacking liver damage. Consequently, the detection of alanine aminotransferase and/or aspartate aminotransferase is of therapeutic importance.

Specific examples of peptide and protein hormone analytes include parathyroid hormone (parathromone), thyrocalcitonin, insulin, glucagon, relaxin, erythropoietin, melanotropin (melanocyte-stimulating hormone and intermedin), somatotropin (growth hormone), corticotropin (adrenocorticotropic hormone), thyrotropin, prolactin, follicle-stimulating hormone, luteinizing hormone), chorionic gonadotropin (hCG), oxytocin, and vasopressin.

Specific examples of polynucleotide analytes include DNA and RNA as well as their nucleoside and nucleotide precursors, which include ATP, NAD, FMN, adenosine, guanosine, thymidine, cytidine, and uracil with their appropriate sugar and phosphate substituents.

Specific examples of vitamin analytes include Vitamin A (i.e. retinol), B (e.g. B₁ or thiamine, B₂ or riboflavin, B₃ or niacin, B₅ or pantothenate, B₆ or pyridoxine, B₇ or biotin, B₉ or folic acid, and B₁₂), C (i.e. ascorbic acid), D (e.g. calciferol, D₂, and D₃), E (i.e. tocopherol), K, and vitamin derivatives or metabolites such as nicotinamide.

Specific examples of microorganism analytes, including infectious disease agents, include corynebacteria, pneumococci, streptococci, staphylococci, neisseriae, hemophilus influenzae, pasteurellae, brucellae, aerobic spore-forming bacilli, anaerobic spore-forming bacilli, mycobacteria, actinomycetes (fungus-like bacteria), the spirochetes, mycoplasmas, and other pathogens, such as listeria monocytogenes, erysipelothrix rhusiopathiae, streptobacillus moniliformis, donvania granulomatis, bartonella bacilliformis, rickettsiae (bacteria-like parasites), fungi, agents causing venereal diseases such as chlamydia, chancroid, granuloma inguinale, gonorrhea, syphilis, jock itch, yeast infection, herpes simplex, HPV, crab louse, scabies, trichomoniasis, and infectious diarrheal microorganisms such as camplylobacter, salmonellae, shigellae, Escherichia coli, Clostridium difficile, Giardia lamblia, Entamoeba histolytica, and organisms causing leptospirosis, nosocomial infections, staphylococcal enterotoxicosis, typhoid fever, cholera, vibrio gastroenteritis, yersinia gastroenteritis, clostridium perfringens gastroenteritis, bacillus cereus gastroenteritis, aflatoxin poisoning, amoebic dysentery, cryptosporidiosis, cyclospora diarrheal infection. Other microorganism analytes include viruses, such as herpes viruses, pox viruses, picornaviruses, myxoviruses (influenza A, B, and C, and mumps, measles, rubella, etc.), arboviruses, reoviruses, rotoviruses, noroviruses, adenoviruses, astroviruses, hepatitis, human immunodeficiency virus, and tumor viruses.

The categories of protein analytes and microorganism analytes may optionally overlap. For example, a microorganism analyte may be detected via the analysis of a protein analyte specific for the microorganism analyte. A protein analyte specific for a microorganism analyte may include an antibody specific for a microorganism analyte, or marker thereof. As a non-limiting example, for a microorganism analyte such as viral hepatitis, antibodies specific to any of viral hepatitis A, B, C, D, E, F and/or G may comprise the protein analyte. Such antibodies include, but are not limited to, immunoglobins such as IgA, IgD, IgE, and specifically IgM and/or IgG, and antibodies to surface antigens, envelope antigens, core antigens, and/or delta antigens (e.g. small and/or large). Specific examples of antigens for viral hepatitis B include hepatitis B surface antigen (HBsAg), hepatitis B envelope antigen (HBeAg), hepatitis B core antigen (HBcAg). Alternatively, a protein analyte specific for a microorganism analyte may include a protein analyte characteristically produced by the microorganism analyte. As a non-limiting example, for a microorganism analyte such as viral hepatitis, proteins specific to any of viral hepatitis A, B, C, D, E, and/or F may comprise the protein analyte. Such protein analytes include, but are not limited to, structural and/or nonstructural proteins. Specific examples of protein analytes for viral hepatitis C include, but are not limited to structural proteins such as E1 and/or E2, and/or nonstructural proteins such as NS2, NS3, NS4, NS4A, NS4B, NS5, NS5A, NS5B, and peptide portions thereof.

The above described analytes possess at least one marker recognized by at least one test reagent and/or signaling reagent. Optionally, the above described analytes may possess multiple markers recognized by the same and/or different test reagents and/or signaling reagents. It is readily envisioned that a marker may be the entire analyte and/or a portion thereof.

A preferred embodiment are testing for analytes detectable through saliva. It is beneficial to test for analytes to aid in the detection of drugs of abuse and thereapeutic drugs, as well as cancer markers, disease markers, hormonal markers, glucose and metabolites.

More specific examples of salivary drug analytes for detecting both drugs of abuse and therapeutic drugs are ethanol, methanol, ethylene glycol, and diethylene glycol.

More specific examples of salivary drug analytes, including both drugs of abuse and therapeutic drugs, include opiates, which includes but is not limited to methadone, morphine, 6-monoacetytl morphine, heroin, dextromethorphen, meperidine, codeine, cocaine, hydromorphone, pholcodine, and metabolites thereof.

Additional salivary drug analytes, including both drugs of abuse and therapeutic drugs, is the barbiturates, such as barbital, amobarbital, hexobarbital, Phenobarbital, methyl phenobarbital, phencyclidine (PCP), pentobarbital.

Additional salivary drug analytes, including both drugs of abuse and therapeutic drugs, includes those derived from marijuana, which includes cannabinol, tetrahydrocannabinol, 11-nor-9-carboxy-delta-9-tetrahydrocannabinol (THC).

More specific examples of salivary drug analytes, including both drugs of abuse and therapeutic drugs, include benzodiazepines including alprazolam, bretazenil, bromazepam, chlorodiazepoxide, cinolazepam, clonazepam, cloxazolam, clorazepate, diazepam, estazolam, fludiazepam, flunirazepam, flurazepam, flutoprazepam, halazepam, ketazolam, loprazolam, lorazepam, lormetazepam, medazepam, midazolam, nimetazepam, nitrazepam, nordiazepam, N-Desmethyldiazepam, oxazepam, phenazepam, pinazepam, prazepam, premazepam, quazepam, temazepam, tetrazepam, triazolam, and other benzodiazepine receptor ligands such as clobazam, DMCM, flumazenil, eszopiclone, zaleplon, zolpidem, and zopiclone. Examples of phenothiazines include chlorpromazine, promethazine, triflupromazine, methotrimeprazine, mesoridazine, thioridazine, fluphenazine, perphenazine, prochlorperazine, and trifluoperazine.

Other individual miscellaneous salivary drug analytes, including both drugs of abuse and therapeutic drugs, include nicotine and cotinine.

Specific examples of salivary analytes used for detecting cancer include mRNA biomarkers for pancreatic cancer, mRNA markers for oral cancer, HER2/neu, CA 15-3, p53, transferrin, cyclin D1, and maspin (serpin B5).

Specific examples of salivary analytes used for detecting certain metabolic disorders include glucose, anti-HIV antibody, HBV surface antigen, anti-HAV (IgM and IgG), anti-Helicobacter pylori (anti-H. pylori IgG), allergen-specific IgA, chromogranin A, lysozyme, peroxidase, hydroxyproline, calcium, and C-reactive protein.

Specific examples of salivary analytes used for detecting hormones include cortisol, alpha amylase, estradiol, progesterone, dehydroepiandrosterone (DHEA), testerosterone, leutinizing hormone, melatonin, and cyclin D.

Specific examples of salivary analytes used for detecting and investigating human psychological phenomenon are cortisol and alpha amylase.

### Samples

An analyte of interest may be present in a wide variety of environments, and it is envisioned that a person having ordinary skill in the art will readily understand that the components and embodiments discussed above can be modified as needed to accommodate different environments of samples.

Analytes of interest may be found in a patient's physiological fluids, such as mucus, blood, serum, blood plasma, lymph, puss, urine, feces, cerebral spinal fluid, ocular lens liquid, ascites, semen, sputum, saliva, sweat, and secreted oils. Samples for testing analytes may be obtained using techniques known or envisioned to provide samples of such physiological fluids. Optionally, analytes may be detected by directly contacting embodiments of the diagnostic test strips with the patient's body, such as their skin, eyes, mouth cavity regions including the tongue, tonsils, and inner lining of the mouth and throat, and the nasal cavity. Alternatively, some analytes may be detected by directly contacting embodiments of the diagnostic test strips with a patient's urine stream, source of bleeding, source of puss, discharge from sex organs, or other site of fluid leakage from the patient.

Analytes may also be found in synthetic chemicals, water, soil, air and food (e.g., milk, meat, poultry, or fish). Any organic- and inorganic-containing substances can serve as an analyte so long as test reagents are available to generate a signal concerning the presence, absence, and/or concentration of the analyte.

For oral fluids such as saliva, samples may be obtained by contacting an embodiment with a patient's tongue such that the tongue contacts the one or more test pads. Alternatively, salivary samples may be obtained by contacting an embodiment with the top and/or sides of a patient's tongue using a substantially back and forth motion from substantially the tip of the tongue to substantially the back of the tongue. Furthermore, salivary samples may be obtained by contacting an embodiment with the top and/or sides of a patient's tongue using a substantially side-to-side motion along the width of the tongue. Similarly, salivary samples may also be obtained by contacting an embodiment with the top and/or sides of a patient's tongue using a substantially circular motion. For each of the above described sample collection methods, the results of the analysis could then be read directly from the diagnostic test strip by a user. Optionally, test results could be stored to a suitable memory device for recordation and later access.

Prior to use with embodiments of the invention, samples may be preserved, stored, or pre-treated in manners consistent with known handling of the same, or similar, types of samples. It is envisioned that any type of preservation, storage, or pre-treatment may be utilized so long as it does not introduce false positives or false negatives into the assay.

### CONCLUSION

While the invention has been described with reference to the specific embodiments thereof, it should be understood by those skilled in the art that various changes may be made and equivalents may be substituted without departing from the true spirit and scope of the invention. For example, some embodiments do not provide all of the benefits and features set forth herein. In addition, many modifications may be made to adapt a particular situation, material, composition of matter, process, process step or steps, to the objective, spirit and scope of the present invention. Furthermore, practiced embodiments may include features of more than one of the described embodiments. All such modifications are intended to be within the scope of the claims appended hereto. Accordingly, the scope of the invention is defined only by reference to the appended claims.

What is claimed is:

1. A diagnostic test strip comprising:
a carrier strip;
one or more test pads on the carrier strip; and
a memory on the carrier strip.

2. The diagnostic test strip of claim 1, wherein the test pads and are not in fluid communication with the memory.

3. The diagnostic test strip of claim 1, wherein the memory is a read-only device.

4. The diagnostic test strip of claim 1, wherein the memory is a rewritable device.

5. The diagnostic test strip of claim 1, wherein there are two or more memories and at least one is a read-only device and at least one is a rewritable device.

6. The diagnostic test strip of claim 1, further comprising a network controller device on the carrier strip and in electrical contact with the memory.

7. The diagnostic test strip of claim 1, further comprising one or more connector pins in electrical contact with the memory.

8. The diagnostic test strip of claim 7, wherein the connector pins are bus connector pins.

9. The diagnostic test strip of claim 7, wherein the connector pins are configured to communicate with a USB data bus.

10. The diagnostic test strip of claim 7, wherein the connector pins are configured to communicate with a serial data bus.

11. The diagnostic test strip of claim 7, wherein the connector pins are configured to communicate with a parallel data bus.

12. The diagnostic test strip of claim 7, wherein the connector pins are configured to communicate with a 1394 data bus.

13. The diagnostic test strip of claim 7, wherein the connector pins are for an Ethernet connection.

14. The diagnostic test strip of claim 1, wherein the memory contains information about one or more of the manufacture date, manufacturer, and indentifying characteristics of any test reagents contained on the one or more test pads.

15. The diagnostic test strip of claim 1, wherein the memory contains information identifying the diagnostic test strip.

16. The diagnostic test strip of claim 1, wherein the memory is an RFID device containing information identifying the diagnostic test strip.

17. The diagnostic test strip of claim 1, wherein at least one of the one or more test pads has a test reagent thereon.

18. The diagnostic test strip of claim 1, wherein there are at least two test pads, each of the test pads contains a different test reagent, and each test reagent detects a different marker on the same analyte.

19. The diagnostic test strip of claim 1, wherein at least one of the one or more test pads further contains a signaling reagent.

20. The diagnostic test strip of claim 1, wherein at least one of the one or more test pads contains a reagent that tests for a saliva-borne analyte.

21. The diagnostic test strip of claim 1, wherein the at least one of the one or more test pads contains a reagent that tests for a sputum-borne analyte.

22. The diagnostic test strip of claim 1, wherein at least one of the one or more test pads contains a reagent that tests for a serum-borne analyte.

23. The diagnostic test strip of claim 1, wherein at least one of the one or more test pads contains a reagent that tests for a plasma-borne analyte.

24. The diagnostic test strip of claim 1, wherein at least one of the one or more test pads contains a reagent that tests for a blood-borne analyte.

25. The diagnostic test strip of claim 1, wherein at least one of the one or more test pads contains a reagent that tests for a urine-borne analyte.

26. The diagnostic test strip of claim 1, wherein at least one of the one or more test pads contains a reagent that tests for a semen-borne analyte.

27. The diagnostic test strip of claim 1, wherein at least one of the one or more test pads contains a reagent that tests for an ascites-borne analyte.

28. The diagnostic test strip of claim 1, wherein at least one of the one or more test pads contains a reagent that tests for a cerebral spinal fluid-borne analyte.

29. A method for detecting one or more analytes in a sample, comprising:
a) contacting the diagnostic test strip of claim 1 with a sample so that the sample contacts the one or more test pads; and
b) reading the results from the test strip.

30. The method of claim 29, further comprising:
reading information from the memory; and
recording the results from the test strip and the information read from the memory in a database.

31. The method of claim 30, wherein the memory is an RF device, and reading the information is performed with an RF device.

32. The method of claim 30, wherein the information read from the memory comprises information identifying the diagnostic test strip.

33. The method of claim 30, wherein recording the results comprises taking a picture of the test pads with a camera, and saving the picture in the database.

34. The method of claim 30, further comprising storing additional information in the database, the additional information being related to one or more of a time of the contacting the diagnostic test strip with the sample, a date of the contacting the diagnostic test strip with the sample, a location where contacting the diagnostic test strip with the sample, an identification of the source of the sample, and identification of an individual administering the detecting method, demographic data of the source of the sample, and physiological data of the source of the sample.

35. The method of claim 29, further comprising:
electronically recording the results from the test strip; and
storing the recorded results in the memory.

36. The method of claim 35, wherein electronically recording the results from the test strip comprises taking a picture of the test pads with a camera.

37. The method of claim 35, further comprising storing additional information in the memory, the additional information being related to one or more of a time of the contacting the diagnostic test strip with the sample, a date of the contacting the diagnostic test strip with the sample, a location where contacting the diagnostic test strip with the sample, an identification of the source of the sample, and identification of an individual administering the detecting method, demographic data of the source of the sample, and physiological data of the source of the sample.

38. The method of claim 29, further comprising contacting the test strip with one or more signaling reagents so that the one or more signaling reagents contact the one or more test pads.

39. The method of claim 29, wherein the patient sample is serum.

40. The method of claim 29, wherein the patient sample is semen.

41. The method of claim 29, wherein the patient sample is urine.

42. The method of claim 41, wherein the test strip is directly contacted with the patient's urine stream.

43. The method of claim 29, wherein the patient sample is saliva.

44. The method of claim 43, wherein the test strip is contacted with patient's tongue.

45. The method of claim 29, wherein the patient sample is blood.

46. The method of claim 45, wherein the test strip is contacted directly with the source of the blood.

47. The method of claim 29, wherein the patient sample is ascites.

48. The method of claim 29, wherein the patient sample is sputum.

49. The method of claim 29, wherein the patient sample is cerebral spinal fluid.
